(19) United States
(12) Patent Application Publication
(10) Pub. No.: US 2004/0098014 A1
(43) Pub. Date: May 20, 2004

(54) Inflatable medical device with combination cutting elements and drug delivery conduits

(76) Inventors: Moshe Flugelman, Haifa (IL); Meir Hefetz, Karashim (IL); Yehoshua Yeshurun, Haifa (IL)

Correspondence Address: Mark Friedman, Bill Polkinghorn, Discovery Dispatch, 9003 Florin Way, Upper Marlboro, MD, 20772, US

(21) Appl. No.: 10/472,960

(22) PCT Filed: Mar. 29, 2002
(86) PCT No.: PCT/IL02/00276

(30) Foreign Application Priority Data
Mar. 30, 2001 (US) 60279704

(51) Int. Cl. A61M 29/00; A61B 17/32

(52) U.S. Cl. 606/192; 606/167

(57) ABSTRACT

The present invention is a balloon angioplasty device which provides a combination of cutting elements to enhance dilation of an artery together with controlled drug delivery directed towards the regions of cutting. The invention is preferably implemented using rows of hollow microneedles to serve both as the cutting elements and the drug delivery conduits. The invention also provides a corresponding method in which a drug is delivered via conduits located within cutting elements around the exterior of a balloon angioplasty device during and/or immediately subsequent to inflation of the balloon within a blood vessel.

### FIELD AND BACKGROUND OF THE INVENTION

The present invention relates to inflatable medical devices and, in particular, it concerns a balloon device which provides a combination of cutting elements to enhance dilation of an artery together with controlled drug delivery directed towards the regions of cutting. The combination is preferably provided by use of rows of hollow microneedles which serve both as the cutting elements and the drug delivery conduit.

It is known to enhance operation of an angioplasty balloon by providing cutting edges deployed to make slight incisions into the stenosis during the angioplasty procedure. An example of a device operating in this manner may be found in U.S. Pat. No. 5,196,024 to Barath and U.S. Pat. No. 5,320,634 to Vigil et al. (Interventional Technologies Inc.), which are both hereby incorporated by reference in their entirety.

It is also known to incorporate drug delivery with an angioplasty balloon. Examples of such devices may be found in U.S. Pat. No. 5,843,033 to Ropiak and U.S. Pat. No. 6,210,392 to Vigil et al. (Interventional Technologies Inc.), which are both hereby incorporated by reference in their entirety.

The Ropiak device provides dispersed delivery of a drug over a large part of the surface of the balloon and does not allow localization of drug delivery to particular regions of importance. As a result, it has been found that the drug is not efficiently absorbed by the tissue and is therefore ineffective. Apparently for this reason, a number of commercial devices based on this technology which were produced by Boston Scientific Corp. (US) have recently been discontinued.

The Vigil et al. ('392) reference teaches an improved configuration in which a drug is delivered by a number of outwardly projecting dispensers which penetrate into the surrounding tissue, thereby minimizing dispersion of the drug. A device based upon this technology is commercially available under the trademark “Infiltrator” from Interventional Technologies Inc. of San Diego (U.S.A.). In this device, three strips each bearing a row of seven needles are positioned around a balloon and are used for injecting a drug into the tissue of a blood vessel wall. Each needle is nearly 0.3 mm long and the spacing between the needles is about 2.5 mm.

It is important to note that the aforementioned technological fields of incision-assisted angioplasty and vascular intra-mural drug delivery have become established as two distinct and independent groups of applications. This is evident, for example, from the contrasting patent documents and corresponding product lines of the aforementioned Interventional Technologies Inc. where common inventors have worked upon both products without at any stage proposing a device for simultaneous incision-assisted angioplasty together with localized intra-mural drug delivery. Amongst other possible reasons, this may be a result of difficulties in implementing drug dispensers as part of a continuous elongated blade. Any attempt to combine the teachings of the above documents directly would require locating the drug dispensers in regions other than where the blades are located, resulting in a device which would deliver a drug primarily to regions other than where the incisions are formed. This is the opposite from the situation which would be preferred clinically in which the drug would be specifically delivered to the region of the incision.

Co-assigned co-pending PCT Patent Publications Nos. WO01/66065 and WO02/17985, which are both hereby incorporated by reference, describe particularly advantageous structures of hollow microneedles which are suitable for transdermal drug delivery or diagnostic sampling. The structures are described therein primarily as two-dimensional arrays of needles on the surface of a wafer.

There is therefore a need for a balloon device for applications such as angioplasty which would provide a combination of cutting elements to enhance dilation of an artery together with controlled drug delivery directed towards the regions of cutting. It would also be highly advantageous to provide such a combination by employing rows of hollow microneedles to serve both as the cutting elements and the drug delivery conduits.

### SUMMARY OF THE INVENTION

The present invention is a balloon angioplasty device which provides a combination of cutting elements to enhance dilation of an artery together with controlled drug delivery directed towards the regions of cutting.

The invention is preferably implemented using rows of hollow microneedles to serve both as the cutting elements and the drug delivery conduits.

The invention also provides a corresponding method in which a drug is delivered via conduits located within cutting elements around the exterior of a balloon angioplasty device during and/or immediately subsequent to inflation of the balloon within a blood vessel.

Thus, according to the teachings of the present invention, there is provided a balloon device comprising: (a) an inflatable balloon inflatable from an uninflated elongated state to an inflated state, the inflated state having a substantially cylindrical enlarged region defining a central axis; and (b) a plurality of elongated cutting configurations associated with the inflatable balloon so as to project from the cylindrical enlarged region in the inflated state, each of the elongated cutting configurations having a direction of elongation substantially parallel to the central axis, each of the elongated cutting configurations including a plurality of drug delivery conduits spaced along the direction of elongation, wherein each of the elongated cutting configurations is configured to cut a substantially continuous incision parallel to the direction of elongation.

According to a further feature of the present invention, each of the elongated cutting configurations includes a plurality of closely spaced microneedles each having a maximum dimension parallel to the direction of elongation, the microneedles being spaced by a distance smaller than the maximum dimension. Preferably, the microneedles are spaced by a distance smaller than half of the maximum dimension.

According to a further feature of the present invention, each of the elongated cutting configurations includes a plurality of microneedles, each of the microneedles having a beveled form including a substantially planar bevel surface, the bevel surface being parallel to the direction of elongation. Preferably, all of the bevel surfaces in each of the elongated cutting configurations lie in a common plane.

According to a further feature of the present invention, each of the drug delivery conduits, is implemented as a conduit formed through one of the microneedles.

### BRIEF DESCRIPTION OF THE DRAWINGS

The invention is herein described, by way of example only, with reference to the accompanying drawings, wherein:

FIG. 1 is a schematic isometric view of a balloon angioplasty device, constructed and operative according to the teachings of the present invention, employing rows of microneedles; and

FIG. 2 is an enlarged isometric view of a row of microneedles for use in the balloon angioplasty device of FIG. 1.

### DESCRIPTION OF THE PREFERRED EMBODIMENTS

The present invention is a balloon angioplasty device which provides a combination of cutting elements to enhance dilation of an artery together with controlled drug delivery directed towards the regions of cutting. The invention is preferably implemented using rows of hollow microneedles to serve both as the cutting elements and the drug delivery conduits. The invention also provides a corresponding method.

In another aspect, by using limited penetration, the invention also provides a highly effective method and device for near-homogeneous distribution of a drug to the tissue of a region of the wall of a biological conduit with minimum trauma to the tissue.

The principles and operation of devices according to the present invention may be better understood with reference to the drawings and the accompanying description, considered in combination with the incorporated references.

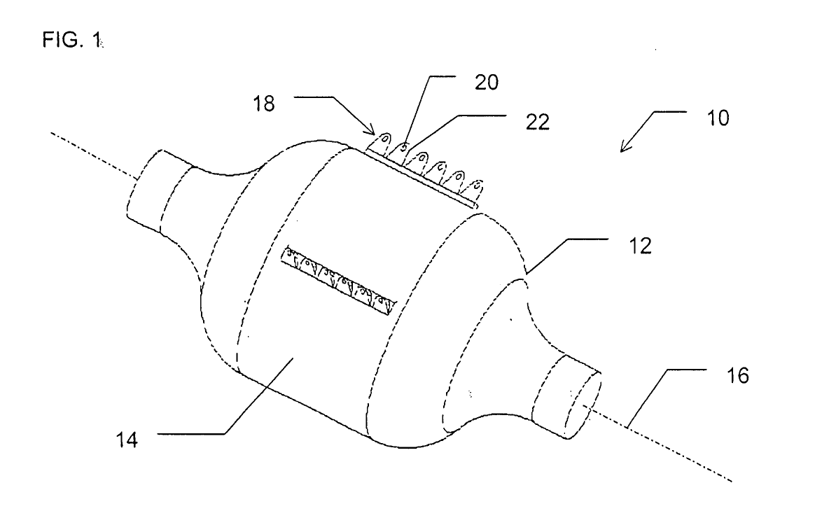

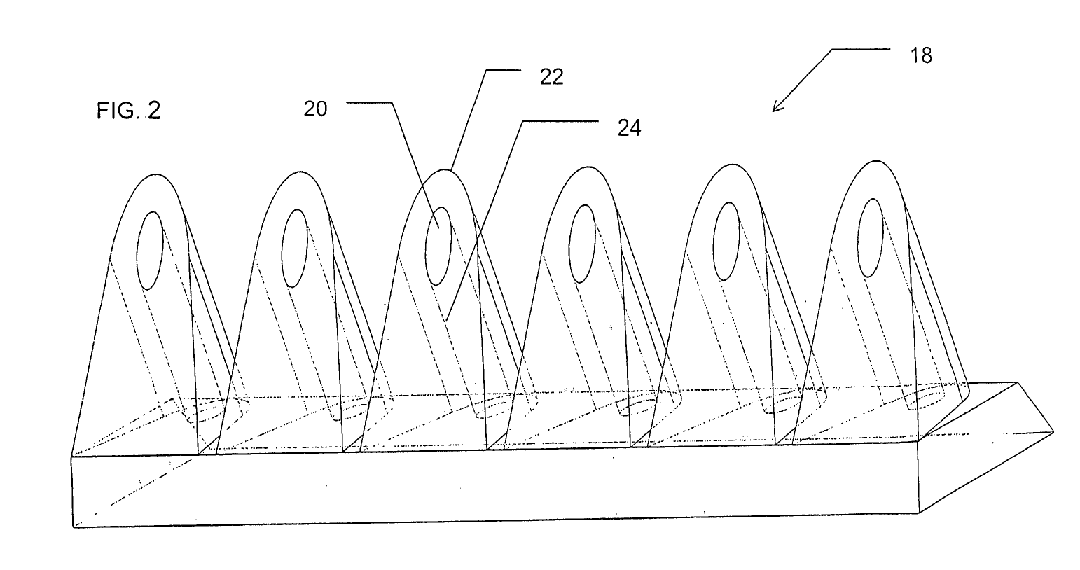

Referring now to the drawings, FIG. 1 shows a balloon device 10, constructed and operative according to the teachings of the present invention. Generally speaking, balloon device 10 has an inflatable balloon 12 inflatable from an uninflated elongated state to an inflated state as shown, the inflated state having a substantially cylindrical enlarged region 14 defining a central axis 16. A plurality of elongated cutting configurations 18 are associated with inflatable balloon 12 so as to project from the cylindrical enlarged region 14 in the inflated state. Each elongated cutting configuration 18 has its direction of elongation substantially parallel to central axis 16. The elongated cutting configurations each include a plurality of drug delivery conduits 20 spaced along its length, and are configured to cut a substantially continuous incision parallel to the direction of elongation. Preferably, elongated cutting configurations 18 are implemented as rows of hollow microneedles 22. A preferred example of such a cutting configuration is shown enlarged in FIG. 2. The microneedles may be produced by a number of production techniques and may assume a corresponding range of forms. Preferably, the microneedles are of a type disclosed in the aforementioned co-assigned and co-pending PCT Patent Publications Nos. WO01/66065 and WO02/17985, and most preferably, as described in the latter of these applications.

The microneedles may be formed from a wide range of bio-compatible materials including, but not limited to, polymeric materials like PMMA (Poly Methyl Meta Acrylate) or Perspex, PC (polycarbonate), super elastic metal alloys such as NiTi, and other metals such as Ti. In the case of polymeric materials, the production techniques described in the aforementioned patent applications may be supplemented by use of hot-embossing or micro-injection molding, as is known in the art. Further details of preferred production techniques may be found in co-assigned and co-pending Israel Patent Application No. 143487 which is hereby incorporated by reference in its entirety.

Preferably, the spacing of the microneedles along the row may is reduced to less than the maximum (e.g., base) dimension of the microneedle along the row (i.e., less than two base widths between centers), and most preferably, to a spacing of no more than about half the base width of the microneedle (i.e. 1½ base widths between centers). This closely-spaced arrangement gives a close approximation to the cutting effect of a continuous blade. This blade-like effect is preferably further enhanced by aligning a bevel plane 24 of the microneedles to be parallel to the extensional direction of the row. Most preferably, the bevel surfaces of each microneedle in a given row lie in a common plane. This results in a structure which closely approximates to a very finely serrated blade.

Parenthetically, it should be noted that the spacing between microneedles in the aforementioned production techniques may be further reduced so that the microneedles run together to form a continuous elongated blade with reduced depth serrations or, using further simplified production techniques, may produce a continuous elongated blade without serrations. The resulting structure of an elongated blade with spaced drug delivery conduits formed therethrough at intervals along its length is a special case of the teachings of the present invention which falls within the broad scope of the present invention.

Although generally not limited to specific dimensions, it should be appreciated that the microneedles of the present invention are provided at significantly higher spatial density than those of the Interventional Technologies Inc. device mentioned above. Additionally, the needles are preferably sharper and penetrate less deeply. This combination of features leads to reduced vascular injury and less endothelial cell denudation, as well as achieving a much more homogeneous distribution of the injected drug than can be achieved by the Interventional Technologies Inc. device.

The following are believed to be indicative of the preferred ranges of dimensions for the microneedle structures. The working or penetration depth is preferably not more than about 200 micrometers, typically at least 100 micrometers, and most preferably 125 to 150 micrometers. The maximum width of the microneedles at the maximum penetrating depth is typically 50 to 100 micrometers and most preferably around 75 micrometers. The total base width of each microneedle is typically between 100 and 200 micrometers, and most preferably around 150 micrometers. The center-to-center spacing along the row between microneedles is preferably not more than 1 mm, more preferably less than 500 micrometers, and most preferably between 150 and 400 micrometers. The total height of each microneedle is typically 150 to 350 micrometers, and most preferably around 250 micrometers. The through hole equivalent diameter is typically in the range between 20 to 50 micrometers, although the hole itself most preferably has an elliptical cross section.

By way of non-limiting example, the device of the present invention may be implemented in a manner generally similar to the device of the aforementioned U.S. Pat. No. 5,196,024 to Barath with the cutting elements replaced by rows of hollow microneedles such as those of FIG. 2. The inflated state of such a structure is represented very schematically and not to scale in FIG. 1. In the collapsed state, the cutting elements are preferably withdrawn between folds of the balloon, as described by Barath. A suitable flexible drug supply line is provided in fluid communication with the conduits at the rear side of the substrate strip upon which the microneedles are formed, as will be clear to one ordinarily skilled in the art. This facilitates direct delivery of the drug into the tissue adjacent to the incisions made by the microneedles. Amongst other options, implementation of the conduit may be in the form of a double concentric balloon structure between which the drug flows, or alternatively, the drug may itself be the primary inflation fluid for the balloon.

The device is described herein as delivering a “drug”. It should be appreciated that the term “drug” is used herein in a broad sense as referring to any liquid or gel material which is employed for its therapeutic or diagnostic effects. Thus, the term “drug” as used herein includes naturally occurring and synthetic medicaments or chemicals, genes and other biological substances.

The suggested device provides a number of advantages over the prior art. Specifically, the balloon comes into close contact with the vessel wall as the needles penetrate into the wall. This close contact prevents the injected material from being distributed in the blood stream due to leakage, as is observed with other local delivery catheters. Furthermore, the device of the present invention delivers the needed material (drugs, genes, etc.) to the region of the incisions in the vessel wall. As a result, the delivered materials penetrating the vessel wall affect the relevant cells and therefore modify cellular and molecular processes that lead to unwanted events such as restenosis.

The catheter may be used to deliver different materials to relevant vascular segments. The delivered materials can be drugs to prevent local thrombosis and neointimal proliferation or DNA plasmids, or viral vectors delivered also for the same purpose. Amongst the advantages of the catheter over other local delivery catheters is that it allows maximal contact with the vascular wall and direct injection to the vascular wall without distribution to the systemic circulation and with less damage than is caused by other catheters such as the Wolinsky catheter. Documentation concerning the extent of vascular damage caused by conventional techniques may be found in: Flugelman M Y, Jaklitsch M T, Newman K D, Casscells S W, Bratthuaer G L, Dichek D A. “Low levels in vivo gene transfer into the arterial wall through a perforated balloon catheter”. Circulation 1992;85:1110-1117.

The materials from which the balloon itself is made are generally standard. Typical examples include, but are not limited to, thermoplastics such as polyurethane, polyvinyl chloride (PVC), polyethylene (PE), polypropylene (PP), polyamides (nylon), and polyesters (PET). Of these, polyurethane is believed to be particularly advantageous for its tailorability to provide required strength, hardness, biostability, thrombogenicity, and chemical resistance properties.

Although the present invention has been described in the context of one preferred set of applications, namely, arterial angioplasty, it should be noted that the structure and method of the present invention are not limited to use in blood vessels and are equally applicable to a wide range of applications in any biological conduit. By way of example, the device of the present invention may be used in the billiary system (inside or outside the liver) for delivery of cytotoxic drugs, or the urinary system, the genital and reproductive system, or the digestive system for similar purposes and other purposes as well.

It will be appreciated that the above descriptions are intended only to serve as examples, and that many other embodiments are possible within the spirit and the scope of the present invention.

What is claimed is:

1. A balloon device comprising:
(a) an inflatable balloon inflatable from an uninflated elongated state to an inflated state, said inflated state having a substantially cylindrical enlarged region defining a central axis; and
(b) a plurality of elongated cutting configurations associated with said inflatable balloon so as to project from said cylindrical enlarged region in said inflated state, each of said elongated cutting configurations having a direction of elongation substantially parallel to said central axis, each of said elongated cutting configurations including a plurality of drug delivery conduits spaced along said direction of elongation,
wherein each of said elongated cutting configurations is configured to cut a substantially continuous incision parallel to said direction of elongation.

2. The balloon device of claim 1, wherein each -of said elongated cutting configurations includes a plurality of closely spaced microneedles each having a maximum dimension parallel to said direction of elongation, said microneedles being spaced by a distance smaller than said maximum dimension.

3. The balloon device of claim 2, wherein said microneedles are spaced by a distance smaller than half of said maximum dimension.

4. The balloon device of claim 2, wherein each of said microneedles has a beveled form including a substantially planar bevel surface, said bevel surface being parallel to said direction of elongation.

5. The balloon device of claim 1, wherein each of said elongated cutting configurations includes a plurality of microneedles, each of said microneedles having a beveled form including a substantially planar bevel surface, said bevel surface being parallel to said direction of elongation.

6. The balloon device of claim 5, wherein all of said bevel surfaces in each of said elongated cutting configurations lie in a common plane.

7. The balloon device of any one of claims 2 to 6, wherein each of said drug delivery conduits is implemented as a conduit formed through one-of said microneedles.

8. A balloon device comprising:
(a) an inflatable balloon inflatable from an uninflated elongated state to an inflated state, said inflated state having a substantially cylindrical enlarged region defining a central axis; and
(b) a plurality of elongated cutting configurations associated with said inflatable balloon so as to project from said cylindrical enlarged region in said inflated state, each of said elongated cutting configurations having a direction of elongation substantially parallel to said central axis, each of said elongated cutting configurations including a plurality of drug delivery conduits spaced along said direction of elongation,
wherein each of said elongated cutting configurations includes a plurality of closely spaced microneedles each having a maximum dimension parallel to said direction of elongation, said microneedles being spaced by a distance smaller than said maximum dimension.

9. The balloon device of claim 8, wherein said microneedles are spaced by a distance smaller than half of said maximum dimension.

10. The balloon device of claim 8, wherein each of said microneedles has a beveled form including a substantially planar bevel surface, said bevel surface being parallel to said direction of elongation.

11. The balloon device of claim 10, wherein all of said bevel surfaces in each of said elongated cutting configurations lie in a common plane.

12. The balloon device of claim 8, wherein each of said drug delivery conduits is implemented as a conduit formed through one of said microneedles.

13. A balloon device comprising:
(a) an inflatable balloon inflatable from an uninflated elongated state to an inflated state, said inflated state having a substantially cylindrical enlarged region defining a central axis; and
(b) a plurality of elongated cutting configurations associated with said inflatable balloon so as to project from said cylindrical enlarged region in said inflated state, each of said elongated cutting configurations having a direction of elongation substantially parallel to said central axis, each of said elongated cutting configurations including a plurality of drug delivery conduits spaced along said direction of elongation,
wherein each of said elongated cutting configurations includes a plurality of microneedles, each of said microneedles having a beveled form including a substantially planar bevel surface, said bevel surface being parallel to said direction of elongation.

14. The balloon device of claim 13, wherein all of said bevel surfaces in each of said elongated cutting configurations lie in a common plane.

15. The balloon device of claim 13, wherein each of said microneedles has a maximum dimension parallel to said direction of elongation, said microneedles being spaced by a distance smaller than said maximum dimension.

16. The balloon device of claim 15, wherein said microneedles are spaced by a distance smaller than half of said maximum dimension.

17. The balloon device of claim 13, wherein each of said drug delivery conduits is implemented as a conduit formed through one of said microneedles.
